(19) United States
(12) Patent Application Publication
(10) Pub. No.: US 2014/0200411 A1
(43) Pub. Date: Jul. 17, 2014

(54) CONDUCTIVE POLYMERS AND USES

(71) Applicant: SurModics, Inc., Eden Prairie, MN (US)

(72) Inventors: Bruce M. Jelle, Chanhassen, MN (US); Aleksey V. Kurdyumov, Maplewood, MN (US); Joseph Schmidt McGonigle, Minneapolis, MN (US); Robert W. Hergenrother, Eden Prairie, MN (US); Nathan A. Lockwood, Minneapolis, MN (US); Joseph Ventura, Elk Grove, CA (US); Sean M. Stucke, Farmington, MN (US)

(73) Assignee: SurModics, Inc., Eden Prairie, MN (US)

(21) Appl. No.: 14/152,667

(22) Filed: Jan. 10, 2014

Related U.S. Application Data
(60) Provisional application No. 61/751,707, filed on Jan. 11, 2013.

(51) Int. Cl. A61L 31/10; A61L 31/14

(52) U.S. Cl. CPC A61L 31/10; A61L 31/14
USPC 600/300; 252/500; 549/50; 525/327.2; 428/419; 428/412; 604/20; 606/33; 607/115; 427/58

(57) ABSTRACT

Disclosed herein are activatable conductive compositions and methods of making and using activatable conductive compositions. In particular, activatable conductive monomers polymers are described and electrically conductive coatings that include activatable conductive polymers.

This application claims the benefit of U.S. Provisional Application No. 61/751,707, filed Jan. 11, 2013, the contents of which are herein incorporated by reference.

### TECHNICAL FIELD

The invention described herein relates to electrically conductive polymers, including methods of making and using electrically conductive polymers.

### BACKGROUND

Conductive polymers are polymers that are able to conduct electricity. Conductive polymers can be used to create thin, electrically conductive polymer coatings and films. These coatings and films have a variety of functional, protective and decorative uses. Although practical applications of conducting polymers were initially limited by intractability and insolubility, 3-substituted thiophenes and aniline derivatives have been prepared which produce processable conductive polymers with a full range of physical and mechanical properties. Typically, conductive polymer coatings and films are prepared by electrodeposition of the polymer on the surface.

Described herein are improved methods of making and using conductive polymers.

### SUMMARY

Described herein is an activatable conductive monomer including a formula:

R—X

wherein R is an activatable group selected from an ultraviolet activatable group, a thermally-activatable group, an activatable silane group, and an activatable mercapto group; and X is an electrically conductive monomer. In one embodiment, X is selected from aniline, pyrrole, thiophene, ethylenedioxythiophene, p-phenylene vinylene, and derivatives, polymers or copolymers thereof. In one embodiment, the activatable group is a photoreactive aryl ketone, for example, acetophenone or benzophenone. In another embodiment, the activatable group is a thermally-reactive group, for example, a carbene generator and a nitrene generator. In a more particular embodiment, the carbene generator is selected from diazirines and diazo-compounds. In another embodiment, the thermally-reactive group is an aryl azide, such as perfluorinated aryl azides, acyl azides and triazolumylides. In another embodiment, the thermally-reactive group includes dioxetane. In another embodiment, the activatable group includes an activatable silane, such as mono-alkoxysilane, di-alkoxysilane, tri-alkoxysilane, and chlorosilane, more particularly, methoxysilane or ethoxysilane. In another embodiment, the activatable group is an activatable mercapto group.

Also described herein are activatable conductive polymers that include one or more activatable groups covalently attached to an electrically conductive polymer. In one embodiment, the electrically conductive polymer is selected from polyfluorenes, polyphenylenes, polypyrenes, polyazulenes, polynaphthalenes, polypyrroles, polycarbazoles, polyindoles, polyazepines, polyanilines, polythiophenes, polyacetylenes, and copolymers thereof. In a more particular embodiment, the electrically conductive polymer is selected from poly(p-phenylene vinylene), poly(3,4-ethylenedioxythiophene) (PEDOT), poly(phenylene vinylene) (PPV), poly(arylene), polyspirobifluorene, poly(3-hexylthiophene), poly(o-methoxyaniline) (POMA), poly(o-phenylenediamine) (PPD), poly(p-phenylene sulfide), and copolymers thereof. In one embodiment, the polymer includes a first end and a single activatable group is covalently bound to the first end of the polymer. In another embodiment, the polymer includes a first end and a second end and two or more activatable groups are covalently bound to the polymer between the first and second end. In one embodiment, the polymer includes between about 25% and about 95% activatable conductive monomers. In another embodiment, the polymer includes at least about 1% activatable conductive monomers. In one embodiment, the activatable group is a photoreactive group, such as an aryl ketone, for example, acetophenone or benzophenone. In another embodiment, the activatable group is a thermally-reactive group. In a more particular embodiment, the thermally-reactive group is a carbene generator or nitrene generator, such as a diazirine or diazo-compound. In one embodiment, the thermally-reactive group is an aryl azide, such as perfluorinated aryl azides, acyl azides and triazolumylides. In another embodiment, the thermally-reactive group includes dioxetane. In another embodiment, the activatable group includes an activatable silane, such as a mono-alkoxysilane, di-alkoxysilane, tri-alkoxysilane, and chlorosilane, for example, methoxysilane or ethoxysilane. In another embodiment, the activatable group is an activatable mercapto group.

In another embodiment, a device is provided, wherein the device includes a surface; and at least one electrically conductive layer on the surface, at least one electrically conductive layer including one or more of the activatable conductive monomers, one or more of the activatable conductive polymers covalently bound to the surface, or a combination thereof. In one embodiment, the surface includes a polymeric surface and, for example, the activatable conductive polymer includes a photoactivatable or thermally-reactive group. In another embodiment, the surface includes a metal surface and, for example, the activatable conductive polymer includes an activatable silane. In another embodiment, the surface includes a glass surface and, for example, the activatable conductive polymer includes an activatable silane. In one embodiment, the conductive polymer layer includes a plurality of end-linked conductive polymers. In one embodiment, the conductive polymer layer includes cross-linked conductive polymers.

In one embodiment, the device includes a medical device configured to transfer electrical energy to a tissue of a patient, for example, a device configured to transfer radiofrequency energy (RF) to the tissue of the patient. In another embodiment, the device includes a medical device configured to sense electrical activity within a body or tissue of a patient. In another embodiment, the device includes a medical device configured to deliver a biologically active agent to a patient. In one embodiment, the medical device is configured to deliver a biologically active agent to a patient by iontophoresis. In one embodiment, the medical device includes one or more electrodes. In another embodiment, the medical device includes one or more sensors.

Also described here are methods of making a conductive polymeric layer on a surface. In one embodiment, the method includes obtaining a first activatable conductive monomer; activating the activatable group on the activatable conductive monomer to covalently bind the activatable conductive monomer to the surface; and polymerizing the covalently bound conductive monomer with one or more second conductive monomers to form a conductive polymer. In one embodiment, one or more of the second conductive monomers include an activatable group. In another embodiment, the second conductive monomers are unactivatable (i.e., do not include an activatable group as defined herein). In another embodiment, the method includes obtaining one or more activatable conductive polymers; activating the activatable group on the conductive polymer to covalently bind one or more conductive polymers to the surface and/or to one or more adjacent polymers.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

FIGS. 1A-F are schematic representations for various monomeric subunits for conductive polymers.

FIGS. 2A-F are schematic representations for various conductive polymers.

FIG. 3 is a schematic representation of an end-linked conductive layer.

FIG. 4 is a schematic representation of a cross-linked conductive layer.

FIG. 5 is a schematic representation of a cross-linked conductive layer.

FIG. 6 is a schematic representation of a method of making a conductive composition, where Nuc is a nucleophile, E is an electrophile and R¹ is an activatable group.

FIG. 7 is a schematic representation of another method of making a conductive composition, where Nuc is a nucleophile, E is an electrophile and R¹ is an activatable group.

FIG. 8 is a schematic representation of a homopolymerization reaction of substituted ethylenedioxythiophene.

FIG. 9 is a schematic representation of a copolymerization reaction of ethylenedioxythiophene with substituted ethylenedioxythiophene.

FIG. 10 is a schematic representation of a copolymerization reaction of thiophene with thiophene containing benzopheneone.

While the invention is susceptible to various modifications and alternative forms, specifics thereof have been shown by way of example and drawings, and will be described in detail. It should be understood, however, that the invention is not limited to the particular embodiments described. On the contrary, the intention is to second modifications, equivalents, and alternatives falling within the spirit and scope of the invention.

### DETAILED DESCRIPTION

Described herein are electrically conductive compositions, including electrically conductive polymers and conductive monomers, methods of making electrically conductive polymers and methods of use. In particular, activatable electrically conductive polymers and activatable conductive monomers are described. The activatable electrically conductive polymers can be used to make electrically conductive coatings that can be covalently attached to a substrate surface to form a thin, durable coating with a high charge density.

### Electrically Conductive Polymers

As used herein, the term “conductive polymer” or “conductive polymeric composition” refers to a polymer or polymeric composition that is capable of conducting an electrical current. In traditional non-conducting polymers, the valence electrons are bound in sp³ hybridized covalent bonds. Such “sigma-bonding electrons” have low mobility and do not contribute to the electrical conductivity of the material. In contrast, conducting polymers generally have a backbone of contiguous sp² hybridized carbon centers. One valence electron on each center resides in a p_{z} orbital, which is orthogonal to the other three sigma-bonds. The electrons in these delocalized orbitals have high mobility, when the material is “doped” by oxidation, which removes some of these delocalized electrons. Thus the p-orbitals form a band, and the electrons within this band become mobile when it is partially emptied. In principle, these same materials can be doped by reduction, which adds electrons to an otherwise unfilled band. In practice, most conductive polymers are doped oxidatively to give p-type materials. Whereas non-conducting polymers generally have a resistivity of greater than about 1000 ohm-meters, conductive polymers have a resistivity of less than about 500 ohm-meters; less than about 250 ohm-meters; less than about 200 ohm-meters; less than about 150 ohm-meters; less than about 125 ohm-meters; or less than about 100 ohm-meters. For example, an electrically conducting formulation such as, but not limited to, polypyrrole or PEDOT, may have a resistivity in the range 1 ohm-meters to 100 ohm-meters.

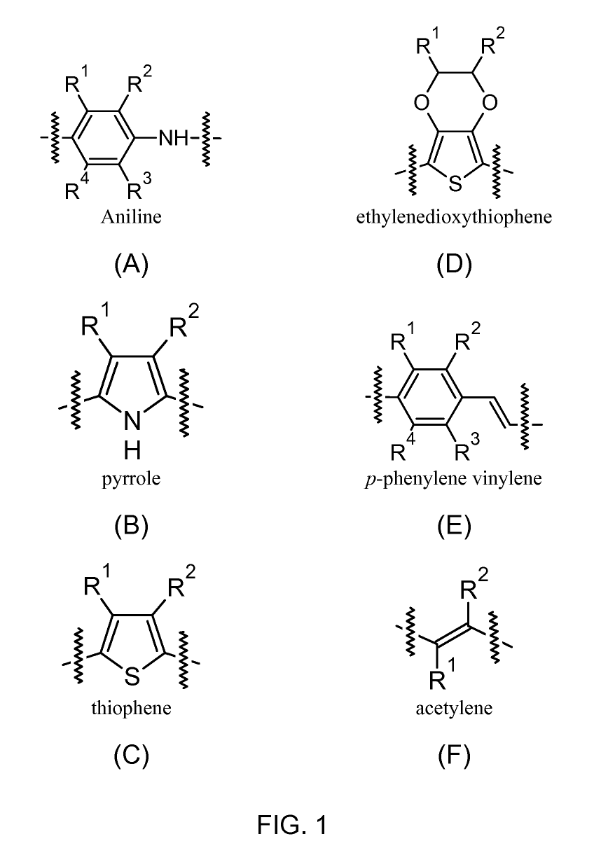

As used herein, the term “organic conductive polymers” refers to polymers that have one or more carbon molecules in the polymer backbone. In one embodiment, the organic conductive polymer has alternating single and double bonds along the polymer backbone. The term “polymer” refers to a class of natural or synthetic macromolecules having 2 or more monomeric subunits. The term “oligomer” refers to short polymer chains, for example, polymer chains having between about 2 and about 20 monomeric subunits. The term “polymer” includes oligomers and longer polymer chains, including, for example, polymer chains having at least about 2, at least about 5, at least about 10, at least about 25, at least about 50 and up to about 100, up to about 250, up to about 500, up to about 1000 or up to about 10,000 or more monomeric subunits. In the case of conductive polymers, individual monomers and oligomers are herein referred to as conductive monomers and conductive oligomers, although it is recognized that, in some instances, the monomeric and/or oligomeric subunits are not, in fact, conductive when they are not incorporated within a polymer. However, for simplicity, individual monomers (i.e., when not included within a polymeric backbone) will nonetheless be referred to as “conductive monomers” and individual oligomers (i.e., when not included within a polymeric backbone) will nonetheless be referred to as “conductive oligomers” herein. When incorporated within a polymeric backbone, the monomers can be referred to as “conductive monomeric subunits.” While in some polymers the monomeric subunits may be the same, in other polymers (herein referred to as copolymers), the monomeric subunits are not all the same or have the same structure. Polymers can include long chains of unbranched or branched monomers and, in some embodiments, can include cross-linked networks of monomers in two or three dimensions. Examples of monomeric subunits for various conductive polymers (also referred to herein as conductive monomers) are shown schematically in FIGS. 1A-F. FIG. 1A: aniline; FIG. 1B: pyrrole; FIG. 1C: thiophene; FIG. 1D: ethylenedioxythiophene; FIG. 1E: p-phenylene vinylene; and FIG. 1F: acetylene; wherein R¹, R², R³ and R⁴ independently are selected from the group consisting of H, alkyl and aryl, with the proviso that (i) in the case of activatable conductive monomers, at least one of R¹ and R² is an activatable group as described herein and (ii) that the alkyl and aryl groups of R¹, R², R³ and R⁴ can be interrupted by a heteroatom. Exemplary R¹, R², R³ and R⁴ groups include, but are not limited to: —(CH₂)ₙOH, —(CH₂)ₙNH₂, —(CH₂)ₙ, C(O)OH, —(CH₂)ₙSi(OR¹¹) and —(CH₂)ₙSH wherein n is an integer in the range from 1-8 and R¹¹ is a C1-C8 alkyl group. These monomers are provided merely as examples. Electrically conductive polymers are not limited to the exemplary monomers listed and can also include copolymers, derivatives and mixtures thereof.

There are many known conductive polymers, including, but not limited to: polymers with aromatic main chains, such as polyfluorenes, polyphenylenes, polypyrenes, polyazulenes, polynaphthalenes; polymers that include nitrogen in the aromatic cycle, such as polypyrroles, polycarbazoles, polyindoles, polyazepines; polymers that include nitrogen outside the aromatic cycle, such as polyanilines; polymers that include sulfur in the aromatic cycle; polymers that include sulfur outside the aromatic cycle, such as poly(p-phenylene sulfide); polymers containing double bonds, such as polyacetylenes; and polymers containing aromatic cycles and double bonds, such as poly(p-phenylene vinylene) and copolymers thereof. Other examples of organic conductive polymers include poly(3,4-ethylenedioxythiophene) (PEDOT), poly(phenylene vinylene) (PPV), poly(arylene), polyspirobifluorene, poly(3-hexylthiophene), poly(o-methoxyaniline) (POMA), and poly(o-phenylenediamine) (PPD). Electrically conductive polymers can also include polymers formed using one or more of the conductive monomers shown schematically in FIGS. 1A-F. However, conductive polymers are not limited to those specifically described herein and can also include mixtures, derivatives, copolymers, mixtures and blends of conductive polymers.

### Activatable Groups

The term “activatable conductive compound” as used herein refers to activatable conductive polymers, activatable conductive oligomers, activatable conductive monomers and combinations thereof. In one embodiment, the activatable conductive compound includes one or more activatable groups. As used herein, the term “activatable” group, which can also be referred to as a “latent reactive group” refers to a moiety that has an unreactive state and a reactive state, wherein the unreactive species responds to an applied external energy in order to undergo active specie generation, resulting in covalent bonding to an adjacent chemical structure, for example, formation of a covalent bond with an adjacent molecule or surface or initiation of polymerization of an adjacent monomer. Activatable groups are preferably sufficiently stable to be stored under conditions in which they retain such properties. See, e.g., U.S. Pat. No. 5,002,582, the disclosure of which is incorporated herein by reference in its entirety. Examples of activatable groups include, but are not limited to photoactivatable groups, thermally-activatable groups, activatable silane groups and other activatable groups such as, mercapto groups. Examples of activatable monomeric subunits for various conductive polymers (also referred to herein as activatable conductive monomers) can be represented schematically by the formula R—X, wherein R is an activatable group selected from a photoactivatable group, a thermally-activatable group, or a reactive silane, and an activatable mercapto group; and X is a conductive monomer. Non-limiting examples of activatable conductive monomers are shown schematically in FIGS. 1A-F, wherein at least one of R¹ and R² is an activatable group as described herein, for example, an activatable group selected from an ultraviolet activatable group, a thermally-activatable group, an activatable silane group, and an activatable mercapto group. Again, these monomers are provided merely as examples of activatable monomers. Activatable monomers can also include derivatives thereof. The term “conductive monomer” as used herein can refer to both conductive monomers that include an activatable group (i.e., activatable conductive monomers) and/or conductive monomers that do not include an activatable group as defined herein (i.e., unactivatable conductive monomers).

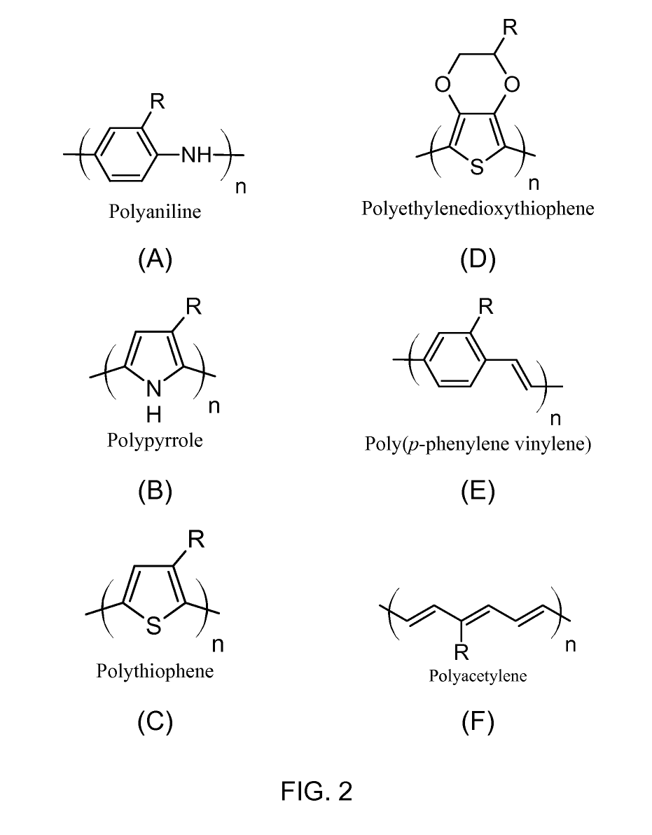

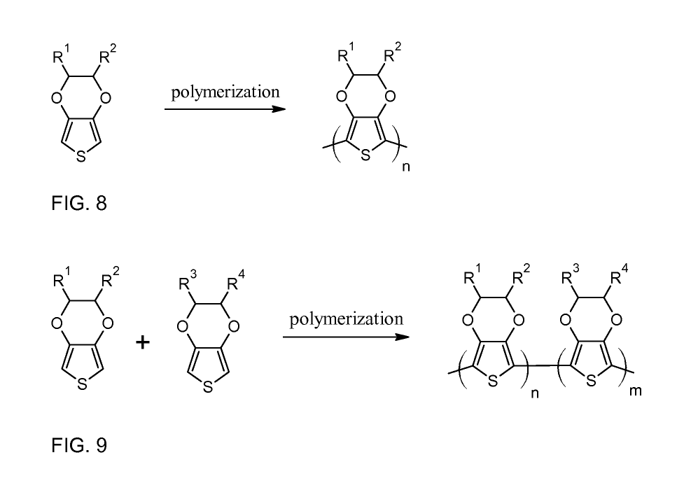

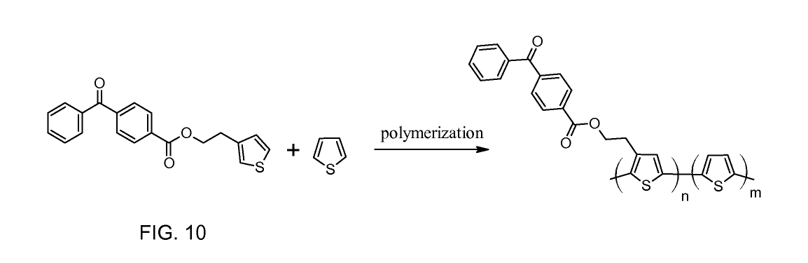

Activatable electrically conductive polymers include one or more activatable groups covalently attached to an electrically conductive polymer. Non-limiting examples of activatable conductive polymers are shown schematically in FIGS. 2A-F, wherein R is an activatable group, for example, an activatable group selected from an ultraviolet activatable group, a thermally-activatable group, an activatable silane group, and an activatable mercapto group. In one embodiment, the monomeric subunits of the electrically conductive polymer each include an activatable group. When the activatable conductive monomers are polymerized, a conductive polymer is formed in which each monomeric subunit includes an activatable group. One example of such a polymerization reaction is shown in FIG. 8, which is a schematic representation of a homopolymerization reaction of substituted ethylenedioxythiophene in which R¹ and R² are independently selected from H, alky, aryl and an activatable group with the proviso that at least one of R¹ and R² is an activatable group and n is an integer between 1 and 10,000. In another embodiment, less than all of the monomeric subunits of the electrically conductive polymer include an activatable group. For example, an activatable conductive copolymer can be formed using both activatable conductive monomers and unactivatable conductive monomers. FIGS. 9 and 10 are a schematic representations of copolymerization reactions of an activatable conductive monomer (i.e., ethylenedioxythiophene in FIG. 9 and thiophene in FIG. 10) and an activatable conductive monomer (i.e., substituted ethylenedioxythiophene in FIG. 9, wherein R¹, R², R³ and R⁴ are independently selected from H, alky, aryl and an activatable group with the proviso that at least one of R¹ and R² is an activatable group, and thiophene containing benzophenone in FIG. 10), wherein n is an integer between 1 and 10,000 and m is an integer between 1 and 10,000. In yet another embodiment, the electrically conductive polymer includes a single activatable group. In a more particular embodiment, the single activatable group is located at a first end of the electrically conductive polymer. In another embodiment, the activatable conductive polymers can include a combination of activatable conductive monomers and conductive monomers without activatable groups (also referred to as unactivatable monomers). In one embodiment, the activatable conductive polymer has a backbone with a first and a second end and includes two or more activatable groups covalently bound to the polymer backbone between the first and second ends. In one embodiment, the activatable conductive polymer includes between about 1% and about 100%; between about 10% and about 90%; or between about 25% and 75% activatable conductive monomers, for example, at least about 1%, 2%, 5%, 10%, 20%, 25%, or 50%, and up to about 75%, 80%, 90%, 95%, 98%, 99% or 100% activatable conductive monomers. Alternately, the composition of the activatable conductive polymer can be expressed as a ratio between activatable conductive monomer and unactivatable conductive monomer subunits. For example, the ratio of activatable conductive monomer and unactivatable conductive monomer can be between 1:100 and 100:1, for example, at least about 1:100; at least about 1:50; at least about 1:10; at least about 1:5; at least about 1:2; or at least about 1:1 and up to about 2:1, up to about 5:1; up to about 10:1, up to about 50:1 or up to about 100:1. The monomeric subunits of the electrically conductive polymer are not limited to the activatable monomers specifically described herein and, additionally, the electrically conductive polymer can include copolymers, derivatives and mixtures of electrically conductive monomers, including activatable and unactivatable conductive monomers.

In one embodiment, the activatable reactive moiety (R group) of the conductive polymer or activatable conductive monomer is responsive to a portion of the electromagnetic spectrum. Activatable reactive groups that are responsive to ultraviolet (UV) and visible portions of the electromagnetic spectrum are referred to herein as “photoreactive” groups. Activatable groups that are responsive to the UV portion of the electromagnetic spectrum can also be referred to as UV activatable.

Photoreactive aryl ketones include, for example, acetophenone and benzophenone, or their derivatives, which are capable of undergoing the activation/inactivation/reactivation cycle described herein. Benzophenone is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source.

Another type of activatable group is a “thermally-reactive group.” The term thermally-reactive refers to a class of compounds that decompose thermally to form reactive species that can form covalent bonds. Both carbenes and nitrenes possess reactive electron pairs that can undergo a variety of reactions, for example, including carbon bond insertion, migration, hydrogen abstraction, and dimerization. Examples of carbene generators include diazirines and diazo-compounds. Examples of nitrene generators include aryl azides, particularly perfluorinated aryl azides, acyl azides, and triazoliumylides. In addition, groups that upon heating form reactive triplet states, such as dioxetanes, or radical anions and radical cations, can be used to as the thermally-reactive group. Generally these compounds thermally decompose at temperatures of not more than 200° C.

In some embodiments the activatable group is a silane group. The term “silane” refers to monomeric silicon compounds. A silane that contains at least one carbon-silicon bond (CH₃—Si) is called an organosilane. Silicon hydride refers to a compound having at least one silicon-hydrogen bond (—Si—H). Silicon hydride is very reactive and can add across carbon-carbon double bonds to form new carbon-silicon bonds. Examples of activatable silane groups include alkoxysilanes, such as mono-, di-, or tri-alkoxysilanes, and methoxysilane or ethoxysilane; and chlorosilanes.

Other activatable groups include activatable mercapto groups.

### Surfaces and Coatings

The activatable conductive compounds described herein can be used to form one or more conductive polymer layers on a surface. In one embodiment, the activatable conductive compounds can be applied to a surface by known electrodeposition techniques. In general, electrodeposited polymers result in the formation of a dark or opaque coating. In another embodiment, in contrast to known conductive polymers, which are almost always electrodeposited on a surface and therefore require that the underlying surface be conductive, the activatable conductive polymers can be applied to and covalently linked to non-conductive surfaces, such as polymeric surfaces. This may be particularly advantageous, because the electrically conductive coating can render a non-conductive surface electrically conductive. Another advantage is that the coating remains clear or transparent (i.e. is not rendered opaque by the electrodeposition process). As such, in other embodiments, the activiatable conductive compounds can be applied to the surface by any conventional method for applying a polymeric coating, including for example, brushing, spray coating or dip coating. In still other embodiments, the activatable polymers can be applied to a surface using both electrodeposition and conventional methods for applying a polymeric coating, such as, brushing, spray coating or dip coating. For example, layers of the coating can be applied in alternating cycles of electrodeposition and conventional methods for applying a polymeric coating, for example, to achieve a coating with the desired properties such as opacity.

In one embodiment, the activatable conductive compounds can be used to coat a polymeric surface. Examples of polymeric surfaces include, for example, hydrophilic polymeric surfaces and hydrophobic polymeric surfaces, crystalline thermoplastics (e.g., high and low density polyethylenes, polypropylenes, acetal resins, nylons and thermoplastic polyesters) and amorphous thermoplastics (e.g., polycarbonates and poly(methyl methacrylates). Examples of polymeric surfaces include, but are not limited to, polystyrene, polycarbonate, polyester, polyethylene, polyethylene terephthalate (PET), polyglycolic acid (PGA), polyolefin, poly-(p-phenyleneterephthalamide), polyphosphazene, polypropylene, polytetrafluoroethylene, polyurethane, polyvinyl chloride, polyacrylate (including polymethacrylate), PEBAX™ and silicone elastomers, as well as copolymers and combinations thereof. In another embodiment, the activatable conductive compounds can be used to coat a glass or metal surface. Glass surfaces can include derivatized glass surfaces, for example, organosilane-pretreated glass, and borosilicate or quartz glasses. Examples of metal surfaces include, for example, precious and non-precious metals such as platinum, gold, stainless steel, nitinol, iridium, titianium and metal oxides and alloys thereof. Other surfaces include silicon hydride surfaces and ceramic surfaces. In one embodiment, an activatable conductive compound includes a photoactivatable or thermally-reactive group and is used to form a layer on a polymeric surface. In another embodiment, the activatable conductive compound includes an activatable silane and is used to form a layer on a metal or glass surface. In one embodiment, the surface is an electrically conductive surface. In another embodiment, the surface is a non-electrically conductive surface, such as a non-conductive polymeric surface.

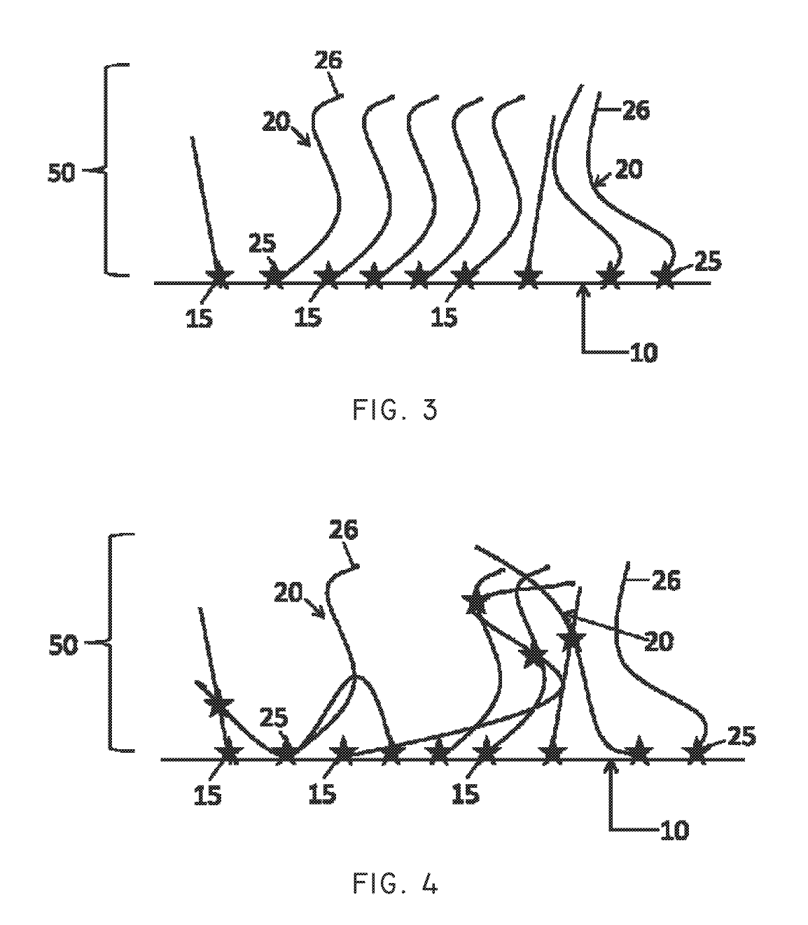

In one embodiment, shown in FIG. 3, the conductive polymer layer 50 includes a plurality of end-linked conductive polymers 20 that are covalently bound to a surface 10. The term “end-linked” refers to a conductive polymer that has a backbone extending between a first 25 and a second 26 end and includes an activatable group 15 proximate the first end 25, such that the conductive polymer layer 50 has a “comb-” or grass-” like configuration (i.e., the conductive polymers 20 do not include additional activatable groups 15 between the first 25 and second 26 ends of the backbone such that the polymers are not crosslinked to one another or the surface 10). In one embodiment, the conductive polymer layer 50 is “grown” on the surface 10 by applying an appropriate energy to activate an activatable group on the activatable conductive monomer, such that a covalent bond is formed between the monomer and the surface. Once the monomer is attached to the surface, subsequent monomers can be added thereto to form a conductive polymer. In one embodiment, the subsequent monomers do not include an activatable group such that the resulting conductive polymer layer is “end-linked” and has the “comb” or “grass” configuration shown in FIG. 3. In another embodiment, shown in FIG. 4, one or more of the subsequent monomers include an activatable group 15, such that the resulting conductive polymer 20 can be crosslinked with neighboring polymers and/or the conductive polymer can be covalently attached to the surface at at least one or at two or more locations along the polymeric backbone. See FIG. 4. In another embodiment, all of the subsequent monomers include an activatable group, such that each monomeric subunit of the polymeric chain includes an activatable group (not shown).

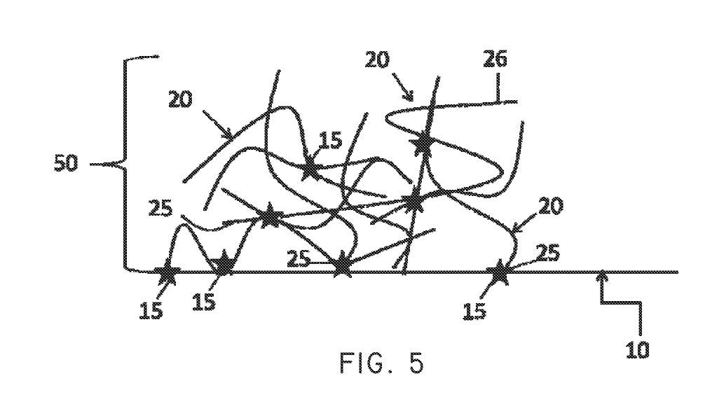

In another embodiment, a conductive polymer layer is formed on a surface by obtaining or preparing an activatable conductive polymer, wherein the polymer includes two or more monomers and one or more activatable groups, and the one or more activatable group on the activatable conductive polymer is/are activated by the application of an appropriate energy to covalently attach the conductive polymer to the surface and/or to one or more adjacent polymers. A cross-linked conductive polymer layer 50 thus formed can be represented schematically as shown in FIG. 5. In the conductive polymer layer 50 shown in FIG. 5, some of the conductive polymers 20 are covalently bound to the surface 10 by one or more activatable groups 15 between a first 25 and second 26 end along the backbone of the conductive polymer 20. In one embodiment, some conductive polymers 20 may be crosslinked to other conductive polymers 20 and to the surface 10 by one or more activatable groups 15 between a first 25 and second 26 end along the backbone of the polymer 20. In a further embodiment, some conductive polymers 20 may be crosslinked to other conductive polymers 20 between a first 25 and a second 26 end along the backbone of the polymer 20, but not directly bound to the surface 10.

### Method of Making

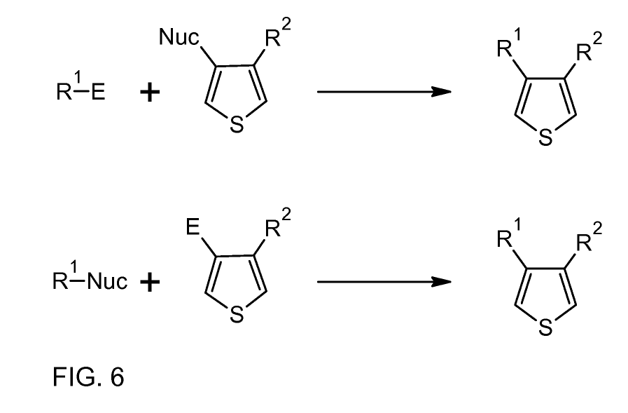

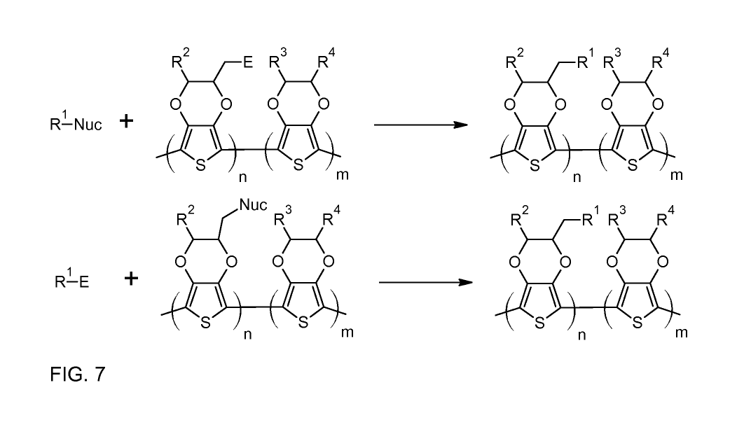

One method of making the activatable conductive compound is shown schematically in FIG. 6, where Nuc is a nucleophile, E is an electrophile and R is an activatable group. In one embodiment, an activatable group (R) containing a suitable electrophile (E) is reacted with a monomer containing a suitable nucleophile (Nuc) under appropriate reaction conditions. In another embodiment, an activatable group (R) containing a suitable nucleophile (Nuc) is reacted with a monomer containing a suitable electrophile (E) under appropriate reaction conditions. Examples of suitable electrophiles include acyl halides such as acid halides, such as acid chlorides, acid fluorides, acid bromides and acid iodides and isocyanate. Examples of suitable nucleophiles include amines, including, but not limited to primary amines, secondary amines, alcohols, and thiols. Examples of suitable reaction conditions include, but are not limited to an anhydrous reaction system or the use of an aprotic solvent or base. Although the schematic shown in FIG. 6 shows the conductive compound as a monomer, the same reaction scheme can be used in the preparation of an activatable conductive polymer, for example, as shown in FIG. 7.

Other exemplary embodiments of methods of making electrically conductive surfaces include grafting polymers onto surfaces. The method can further include a grafting reagent, using the reagent to form a conductive polymeric surface on a support surface or substrate. Methods and materials for grafting polymers onto surfaces are described in U.S. Pat. No. 7,087,658 (Swan, et al.); U.S. Pat. No. 7,348,055 (Chappa, et al.) and U.S. Pat. No. 7,736,689 (Chappa, et al.), the disclosures of which are all hereby incorporated by reference in their entirety.

### Applications

In one embodiment, an article including at least one electrically conductive layer is provided, wherein the electrically conductive layer includes one or more of the conductive compounds described herein. For example, a conductive polymeric layer can be applied to a surface of a device used for transferring electrical energy or a sensor for detecting electrical energy. In one embodiment, a conductive polymer layer can be applied to a surface of a medical device used to transfer electrical energy to a tissue of a patient. In another embodiment, a conductive polymer layer can be applied to a surface of a medical device used to sense electrical activity within the body or tissue of a patient. In still another embodiment, a conductive polymer layer can be applied to a surface of a medical device used to deliver a biologically active agent to a patient, for example, by iontophoresis. In one embodiment, the conductive layer is applied to the surface of a device used to transfer energy to a tissue of a patient, including, for example, a medical device that uses radiofrequency energy (RF). Examples of medical devices that use radiofrequency energy (RF), include a probe used for vein ablation for the treatment of venous insufficiency (i.e., varicose veins); renal denervation to treat hypertension by the application of radio frequency (RF) energy to disrupt hyperactive nerves; other surgical ablation instruments, for example, for the treatment of uterine fibroids; esophagus; chronic obstructive pulmonary disease (COPD); lung cancer; urogenital for incontinence.

Other medical devices that transfer energy to one or more tissues of a patient includes electrodes, such as blood gas electrodes, defibrillator electrodes, electrocardiogram (ECG) electrodes, electrosurgical electrodes, fetal scalp electrodes, ion-selective electrodes, pH electrodes, nasopharyngeal electrodes, pacemaker electrodes, and transcutaneous electrical nerve stimulation (TENS) electrodes.

In another embodiment, the conductive polymeric coating is applied to a surface of a sensor, including for example, sensors for detecting biological materials in a patient (biologic sensors), for example, nitric oxide synthase, peroxynitrite, superoxide, NADH, thrombin, DNA, glutamate, heavy metal ions, etc.; sensors to detect electrical energy in a patient, such as electrodes; or other sensors, such as fiber optic sensor, acoustic sensor, infrared sensor, and the like.

In another embodiment, a conductive polymeric coating can be applied to the surface of a medical device used for drug delivery, for example, a medical device used for iontophoresis in which a small electric charge is used to deliver one or more active agents to one or more tissues of a patient, for example, for transdermal delivery of one or more active agents. In another embodiment, a conductive polymeric coating can be used in connection with implantable electronic active agent delivery systems, for example, for electrically-modulated drug delivery, for example, to administer active agents to fight bacterial infection, reduce inflammation, promote bone growth or reduce fibroblast function.

Other embodiments of conductive polymer coatings described herein can be used on medical devices for cardiac pacing, cell therapy, electroporation and nerve regeneration.

In treatments for cardiac pacing cardiac cells are derived from stem cells, progenitor cells or other types of cells engineered to repair heart function. These cells are then delivered to the region of damaged heart tissue to restore function sometimes inside a matrix or scaffold to retain the cells at the injection site. One of the challenges with this type of therapy is getting the delivered cells to beat in sync with the native heart tissue and an electroconductive polymer matrix or coating included with the delivered cells may provide a conduit for improved electrical integration with the native tissue. Another application can be in the delivery of engineered pacemaker cells which would also benefit from the use of a scaffold made from electroconductive polymers or coated with them to improve their connection to the native heart tissue. Scaffolds may also need to include peptides or other bioactive cues to promote cell survival.

Electroporation is the use of an electric field to create temporary holes in cell membranes such that large molecules such as DNA can pass through the membrane. This can be used to modify cells ex vivo but could also be used in vivo to deliver drugs or help drug permeate barrier membranes such as the blood brain barrier. Electroconductive polymer coatings can be useful on Electroporation devices to improve the efficiency of electric field delivery both ex vivo and in vivo.

Electrical stimulation has been shown to be useful for regeneration of nerves where it promotes the growth of axons. Electroconductive coatings can be useful to enhance the performance of devices designed to stimulate nerve growth by improving the tissue interface. Electroconductive coatings can also be useful as part of scaffolds intended to guide nerve regeneration. In this setting the coatings may also need to include peptides or other bioactive cues to guide nerve growth. Terms used herein have meanings as commonly understood by a person of ordinary skill in the areas relevant to the present invention.

Other medical devices using electrconductive coatings of the compositions described herein include, but are not limited to deep brain stimulation for treatment of tremors, Parkinsons disease and dystonia. Additionally, medical devices for treating OCD, depression, tinnitus, epilepsy, stroke, pain, Alzheimers disease, respiratory support, chronic pain, spasticity, ALS, Huntington's disease, obesity, gastroparesis, irritable bowel syndrome, profound deafness, headaches, traumatic brain injuries, epilepsy, angina pain, incontinence, pelvic pain and sexual dysfunction are included.

Terms such as “a,” “an,” and “the” are not intended to refer to only a singular entity, but include the general class of which a specific example may be used for illustration. The terminology herein is used to describe specific embodiments of the invention, but their usage does not limit the invention, except as outlined in the claims.

In the specification and claims, the term “about” is used to modify, for example, the quantity of an ingredient in a composition, concentration, volume, process temperature, process time, yield, flow rate, pressure, and like values, and ranges thereof, employed in describing various embodiments. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

All publications and patent applications in this specification are indicative of the level of ordinary skill in the art to which this invention pertains. All publications and patent applications are herein incorporated by reference to the same extent as if each individual publication or patent application was specifically and individually indicated by reference. To the extent inconsistencies arise between publications and patent applications incorporated by reference and the present disclosure, information in the present disclosure will govern.

The invention has been described with reference to various specific and preferred embodiments and techniques. However, it should be understood that many variations and modifications may be made while remaining within the spirit and scope of the invention.

### WORKING EXAMPLES

### Example 1

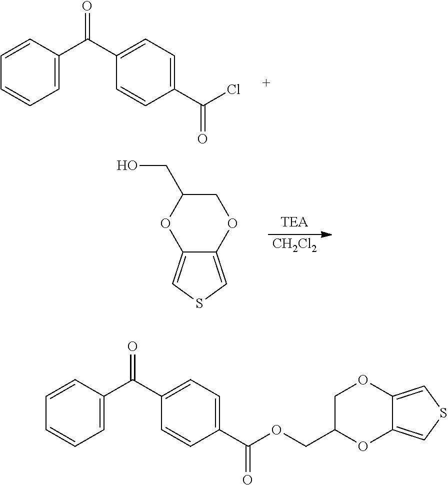

One method for preparing an activatable conductive compound includes dissolving 4-Benzoylbenzoic chloride (1 eqiuv) in methylene chloride under anhydrious conditions. triethylamine (TEA) (1.2 equiv) is added followed by addition of hydroxymethyl-3,4-ethylenedioxythiophene (1 equiv). The reaction is allowed to stir at ambient temperature and is then poured into water, organic layer is collected, washed with sat aq NaHCO₃, water and sat aq NaCl, dried over Na₂SO₄ and filtered. The solvent is reduced and the residue is purified by crystallization or chromatography to afford the desired monomer.

### Example 2

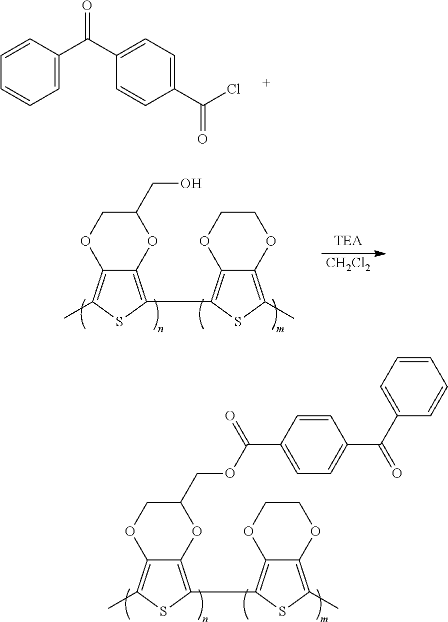

Another method for preparing an activatable conductive compound includes reacting hydroxy-containing polyethylenedioxythiophene with benzoylbenzoic chloride in presence of triethylamine under anhydrious conditions to give benzophenone-substituted polyethylenedioxythiophene.

1. An activatable conductive monomer comprising a formula:
R—X
wherein R is an activatable group selected from an ultraviolet activatable group, a thermally-activatable group, an activatable silane group and an activatable mercapto group; and X is an electrically conductive monomer.

2. The activatable conductive monomer of claim 1, wherein X is selected from aniline, pyrrole, thiophene, ethylenedioxythiophene, p-phenylene vinylene, and derivatives, polymers or copolymers thereof.

3. The activatable conductive monomer of claim 1, wherein the activatable group is a photoreactive aryl ketone.

4. (canceled)

5. The activatable conductive monomer of claim 1, wherein the activatable group is a thermally-reactive group.

6-10. (canceled)

11. The activatable conductive monomer of claim 1, wherein the activatable group comprises an activatable silane.

12. The activatable conductive monomer of claim 11, wherein the activatable silane is selected from mono-alkoxysilane, di-alkoxysilane, tri-alkoxysilane, and chlorosilane.

13-33. (canceled)

34. A device comprising:
a surface; and
at least one electrically conductive layer on the surface, at least one electrically conductive layer comprising one or more of the activatable conductive monomers of any one of claims 1-14; one or more of the activatable conductive polymers of any one of claims 15-33 covalently bound to the surface; or a combination thereof.

35. The device according to claim 34, wherein the surface comprises a polymeric surface.

36. The device according to claim 35, wherein the polymeric surface is selected from polystyrene, polycarbonate, polyester, polyethylene, polyethylene terephthalate (PET), polyglycolic acid (PGA), polyolefin, poly-(p-phenyleneterephthalamide), polyphosphazene, polypropylene, polytetrafluoroethylene, polyurethane, polyvinyl chloride, polyacrylate (including polymethacrylate), silicone elastomers, and copolymers and combinations thereof.

37. The device according to claim 35, wherein the activatable conductive polymer comprises a photoactivatable or thermally-reactive group.

38. The device according to claim 34, wherein the surface comprises a metal surface.

39. (canceled)

40. The device according to claim 38, wherein the activatable conductive polymer comprises an activatable silane.

41. The device according to claim 34, wherein the surface comprises a glass surface.

42. (canceled)

43. The device according to claim 41, wherein the activatable conductive polymer comprises an activatable silane.

44. The device according to claim 43, wherein the conductive polymer layer comprises a plurality of end-linked conductive polymers.

45. (canceled)

46. The device according to claim 34, wherein the device comprises a medical device configured to transfer electrical energy to a tissue of a patient.

47. (canceled)

48. The device according to claim 34, wherein the device comprises a medical device configured to sense electrical activity within a body or tissue of a patient.

49-52. (canceled)

53. A method of making a conductive polymeric layer on a surface, the method comprising:
a. obtaining a first activatable conductive monomer according to claim 1;
b. activating the activatable group on the activatable conductive monomer to covalently bind the activatable conductive monomer to the surface; and
c. polymerizing the covalently bound conductive monomer with one or more second conductive monomers to form a conductive polymer.

54. The method of claim 53, wherein one or more of the second conductive monomers include an activatable group.

55. The method according to claim 53, wherein the second conductive monomers are unactivatable.

56. (canceled)
